(19) United States
(12) Patent Application Publication
(10) Pub. No.: US 2025/0000433 A1
(43) Pub. Date: Jan. 2, 2025

(54) PREFERENCE DETERMINATION APPARATUS, PREFERENCE DETERMINATION METHOD, AND NON-TRANSITORY STORAGE MEDIUM

(71) Applicant: JVCKENWOOD Corporation, Yokohama-shi (JP)

(72) Inventors: Takayuki Sugahara, Yokohama-shi (JP); Shohei Odan, Yokohama-shi (JP); Hideo Tsuru, Yokohama-shi (JP); Kazuki Horikiri, Yokohama-shi (JP)

(21) Appl. No.: 18/830,652

(22) Filed: Sep. 11, 2024

Related U.S. Application Data
(63) Continuation of application No. PCT/JP2023/009021, filed on Mar. 9, 2023.

(30) Foreign Application Priority Data
Mar. 15, 2022 (JP) 2022-040312

(51) Int. Cl. A61B 5/00

(52) U.S. Cl. CPC A61B 5/4035; A61B 5/0048; A61B 5/7264

(57) ABSTRACT

A preference determination apparatus includes: a stimulation unit configured to apply a visual, a tactile, and an auditory stimuli; an activity calculation unit configured to calculate degrees of autonomic nervous activity based on biological signals for the multiple stimuli respectively; a storage configured to store thresholds of autonomic nervous activity set in advance for the multiple stimuli respectively; and a determination unit configured to determine preference by comparing the calculated degrees of autonomic nervous activity with the corresponding stored thresholds, wherein the determination unit is further configured to determine that the preference is high when each of the calculated degrees of autonomic nervous activity for each of the stimuli is larger than each of the corresponding stored thresholds therefor, and when the calculated degree of autonomic nervous activity for the stimuli applied simultaneously is larger than the corresponding stored threshold therefor.

### CROSS-REFERENCE TO RELATED APPLICATIONS

This application is a Continuation of PCT International Application No. PCT/JP2023/009021 filed on Mar. 9, 2023 which claims the benefit of priority from Japanese Patent Application No. 2022-040312 filed on Mar. 15, 2022, the entire contents of both of which are incorporated herein by reference.

### FIELD OF THE INVENTION

The present application relates to a preference determination apparatus, a preference determination method, and a non-transitory storage medium.

### BACKGROUND OF THE INVENTION

There is a technique for evaluating emotions, feelings, and satisfaction level of a person by measuring brain waves of the person while the person is receiving visual and auditory stimuli or doing work that is psychologically burdensome. Such a technique is described in, for example, Japanese Laid-open Patent Publication No. H10-262942.

Senses of a human brain for preference originate from an overall activity of the brain for the five senses (sense of sight, sense of hearing, sense of taste, sense of smell, and sense of touch). Influence on senses for preference of a person is increased under specific conditions. A method to evaluate the senses for the preference is thus need to be creative.

### SUMMARY OF THE INVENTION

A preference determination apparatus, a preference determination method, and a non-transitory storage medium are disclosed.

According to one aspect of the present application, there is provided a preference determination apparatus, comprising: a stimulation unit configured to apply a visual stimulus, a tactile stimulus, and an auditory stimulus; an activity calculation unit configured to calculate degrees of autonomic nervous activity based on biological signals for the multiple stimuli respectively; a storage configured to store thresholds of autonomic nervous activity set in advance for the visual stimulus, the tactile stimulus, and the auditory stimulus respectively; and a determination unit configured to determine preference by comparing the degrees of autonomic nervous activity calculated by the activity calculation unit with the corresponding thresholds stored in the storage, wherein the stimulation unit is further configured to apply each of the multiple stimuli at predetermined intervals, the activity calculation unit is further configured to calculate the degree of autonomic nervous activity based on the biological signal for each of the multiple stimuli applied by the stimulation unit, the storage is further configured to store the threshold of autonomic nervous activity for each of the multiple stimuli, and the determination unit has a first determination unit configured to determine the preference by comparing the multiple degrees of autonomic nervous activity calculated by the activity calculation unit with the corresponding thresholds stored in the storage, the stimulation unit is further configured to apply the multiple stimuli simultaneously, the activity calculation unit is further configured to calculate the degree of autonomic nervous activity based on the biological signal for the multiple stimuli simultaneously applied by the stimulation unit, the storage is further configured to store the threshold of autonomic nervous activity for the multiple stimuli simultaneously applied, and the determination unit has a second determination unit configured to determine the preference by comparing the degree of autonomic nervous activity calculated by the activity calculation unit with the corresponding threshold stored in the storage, and the determination unit is further configured to determine that the preference is high when each of the degrees of autonomic nervous activity calculated based on the biological signal for each of the stimuli applied by the stimulation unit is larger than each of the corresponding thresholds of autonomic nervous activity stored in the storage, and when the degree of autonomic nervous activity calculated based on the biological signal for the stimuli simultaneously applied by the stimulation unit is larger than the corresponding threshold of autonomic nervous activity stored for the multiple stimuli simultaneously applied.

According to one aspect of the present application, there is provided a preference determination method, comprising: applying a visual stimulus, a tactile stimulus, and an auditory stimulus; calculating degrees of autonomic nervous activity based on biological signals for the applied stimulus; storing thresholds of autonomic nervous activity that have been set in advance for the visual stimulus, the tactile stimulus, and the auditory stimulus respectively; and determining preference by comparing the calculated degrees of autonomic nervous activity with the corresponding stored thresholds, further comprises: applying each of the multiple stimuli at predetermined intervals; calculating the degree of autonomic nervous activity based on the biological signal for each of the multiple stimuli applied by the stimulation unit; storing the threshold of autonomic nervous activity for each of the multiple stimuli; and determining the preference by comparing the calculated multiple degrees of autonomic nervous activity with the corresponding stored thresholds, and applying the multiple stimuli simultaneously; calculating the degree of autonomic nervous activity based on the biological signals for the multiple stimuli simultaneously applied; storing the threshold of autonomic nervous activity for the multiple stimuli simultaneously applied; and determining the preference by comparing the calculated degree of autonomic nervous activity with the corresponding stored threshold, and determining that the preference is high when each of the degrees of autonomic nervous activity calculated based on the biological signal for each of the applied stimuli is larger than each of the corresponding thresholds of autonomic nervous activity stored in the storage, and also when the degree of autonomic nervous activity calculated based on the biological signal for the stimuli simultaneously applied is larger than the corresponding threshold of autonomic nervous activity stored for the multiple stimuli simultaneously applied.

According to one aspect of the present application, there is provided a non-transitory storage medium that stores a program that causes a computer, which operates as a preference determination apparatus, to execute: a step of applying a visual stimulus, a tactile stimulus, and an auditory stimulus; a step of calculating degrees of autonomic nervous activity based on biological signals for the applied stimulus; a step of storing thresholds of autonomic nervous activity that have been set in advance for the visual stimulus, the tactile stimulus, and the auditory stimulus respectively; and a step of determining preference by comparing the calculated degrees of autonomic nervous activity with the corresponding stored thresholds, further comprises: a step of applying each of the multiple stimuli at predetermined intervals; a step of calculating the degree of autonomic nervous activity based on the biological signal for each of the multiple stimuli applied by the stimulation unit; a step of storing the threshold of autonomic nervous activity for each of the multiple stimuli; and a step of determining the preference by comparing the calculated multiple degrees of autonomic nervous activity with the corresponding stored thresholds, and a step of applying the multiple stimuli simultaneously; a step of calculating the degree of autonomic nervous activity based on the biological signals for the multiple stimuli simultaneously applied; a step of storing the threshold of autonomic nervous activity for the multiple stimuli simultaneously applied; and a step of determining the preference by comparing the calculated degree of autonomic nervous activity with the corresponding stored threshold, and a step of determining that the preference is high when each of the degrees of autonomic nervous activity calculated based on the biological signal for each of the applied stimuli is larger than each of the corresponding thresholds of autonomic nervous activity stored in the storage, and also when the degree of autonomic nervous activity calculated based on the biological signal for the stimuli simultaneously applied is larger than the corresponding threshold of autonomic nervous activity stored for the multiple stimuli simultaneously applied.

According to one aspect of the present application, there is provided a preference determination apparatus, comprising: a stimulation unit configured to apply a visual stimulus, a tactile stimulus, and an auditory stimulus; an activity calculation unit configured to calculate degrees of autonomic nervous activity based on biological signals for the multiple stimuli respectively; a storage configured to store thresholds of autonomic nervous activity set in advance for the visual stimulus, the tactile stimulus, and the auditory stimulus respectively; and a determination unit configured to determine preference by comparing the degrees of autonomic nervous activity calculated by the activity calculation unit with the corresponding thresholds stored in the storage, wherein the stimulation unit is further configured to apply each of the multiple stimuli at predetermined intervals, the activity calculation unit is further configured to calculate the degree of autonomic nervous activity based on the biological signal for each of the multiple stimuli applied by the stimulation unit, the storage is further configured to store the threshold of autonomic nervous activity for each of the multiple stimuli, and the determination unit has a first determination unit configured to determine the preference by comparing the multiple degrees of autonomic nervous activity calculated by the activity calculation unit with the corresponding thresholds stored in the storage, the stimulation unit is further configured to apply the multiple stimuli simultaneously, the activity calculation unit is further configured to calculate the degree of autonomic nervous activity based on the biological signal for the multiple stimuli simultaneously applied by the stimulation unit, the storage is further configured to store the threshold of autonomic nervous activity for the multiple stimuli simultaneously applied, and the determination unit has a second determination unit configured to determine the preference by comparing the degree of autonomic nervous activity calculated by the activity calculation unit with the corresponding threshold stored in the storage, and the determination unit is further configured to determine that the preference is high when any of the degrees of autonomic nervous activity calculated based on the biological signal for each of the stimuli applied by the stimulation unit is not larger than the corresponding threshold of autonomic nervous activity stored in the storage, and when the degree of autonomic nervous activity calculated based on the biological signal for the stimuli simultaneously applied by the stimulation unit is larger than the corresponding threshold of autonomic nervous activity stored for the multiple stimuli simultaneously applied.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram of a preference determination apparatus according to a first embodiment;

FIG. 2 is a graph depicting physiological characteristics of a biological signal;

FIG. 3 is a schematic diagram illustrating autonomic nervous activity;

FIG. 4 is a schematic diagram illustrating an activity threshold map used for preference determination;

FIG. 5 is a schematic diagram illustrating a first modified example of the activity threshold map;

FIG. 6 is a schematic diagram illustrating a second modified example of the activity threshold map;

FIG. 7 is a schematic diagram illustrating a third modified example of the activity threshold map;

FIG. 8 is a flowchart illustrating a method of calculating a degree of activity;

FIG. 9 is a flowchart illustrating a preference determination method;

FIG. 10 is a block diagram of a preference determination apparatus according to a second embodiment;

FIG. 11 is a schematic diagram illustrating an activity threshold map used for preference determination;

FIG. 12 is a flowchart illustrating a method of calculating a degree of activity;

FIG. 13 is a flowchart illustrating a preference determination method; and

FIG. 14 is a flowchart illustrating a modified example of the preference determination method.

### DETAILED DESCRIPTION OF THE INVENTION

Embodiments of a preference determination apparatus, a preference determination method, and a program, according to the present application will hereinafter be described in detail by reference to the appended drawings. The present invention is not to be limited by the following embodiments.

### First Embodiment

### Preference Determination Apparatus

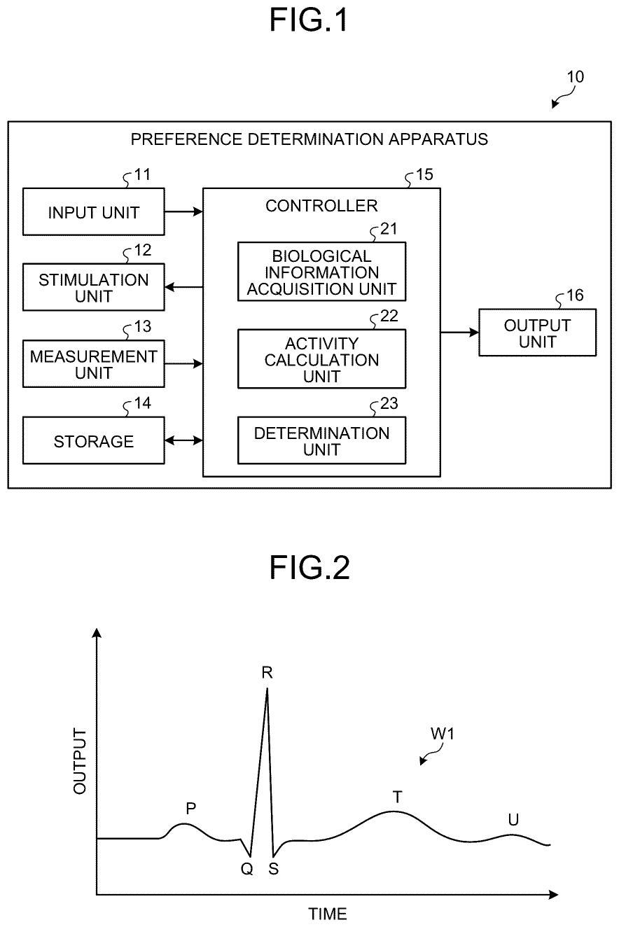

FIG. 1 is a block diagram of a preference determination apparatus according to a first embodiment.

As illustrated in FIG. 1, a preference determination apparatus 10 includes an input unit 11, a stimulation unit 12, a measurement unit 13, a storage 14, a controller 15, and an output unit 16.

The input unit 11 is connected to the controller 15. The input unit 11 is able to be operated by a user and enables input of various signals to the controller 15. The input unit 11 inputs, for example, a start signal for starting a preference determination for a user and an end signal for ending the preference determination for the user, to the controller 15. The input unit 11 is able to be implemented by, for example, a touch panel, buttons, switches, and a keyboard.

The stimulation unit 12 is connected to the controller 15. The stimulation unit 12 is capable of applying multiple stimuli to a user. The controller 15 provides a stimulus application signal to the stimulation unit 12 based on a program. Based on the stimulus application signal input from the controller 15, the stimulation unit 12 applies stimuli to a user. The stimulation unit 12 applies stimuli of multiple levels to a predetermined number of sense organs of the five senses. The multiple levels refer to strength levels of the stimuli. In this case, the stimulation unit 12 applies multiple stimuli to the user at predetermined intervals.

Types of stimuli for a user correspond to, for example, sense of sight, sense of hearing, sense of touch, sense of smell, and sense of taste of the five senses. A stimulus by the sense of sight (hereinafter called a visual stimulus) is a stimulus of letting the user see a video. A stimulus by the sense of hearing (hereinafter called a auditory stimulus) is a stimulus of letting the user hear audio. A stimulus by the sense of touch (hereinafter called a tactile stimulus) is a stimulus of letting the user touch an object. A stimulus by the sense of smell (hereinafter called an olfactory stimulus) is a stimulus of letting the user smell a smell (fragrance). A stimulus by the sense of taste (hereinafter called a gustatory stimulus) is a stimulus of letting the user eat food.

A level of the visual stimulus is, for example, a size (area) of a video to be seen by a user. A level of the auditory stimulus is loudness of audio to be heard by the user. A level of the tactile stimulus is a size (contact area) to be touched by the user. A level of the olfactory stimulus is strength of a smell (fragrance) to be smelt by the user. A level of the gustatory stimulus is strength of a taste of food to be eaten by the user.

Specifically, the stimulation unit 12 serves as a device to apply the visual, auditory, tactile, olfactory, and gustatory stimuli corresponding to the five senses to a user. For example, the visual stimulus is given by a display, the auditory stimulus is given by a speaker or headphones, the tactile stimulus is given by a vibrator or a heating device, and the olfactory or gustatory stimulus is given by a device that discharges a specific smell or fragrance from a dedicated cartridge.

Types of stimuli to be applied to a user are not limited to these types corresponding to the five senses. The stimulation unit 12 applies stimuli of multiple levels for multiple different senses for one of sense organs corresponding to the five senses. The multiple different senses for one of the sense organs mean, for example, audio of different sound qualities for a specific sound for the sense of hearing. For the sense of taste, they correspond to, for example, sweetness, sourness, saltiness, bitterness, and umami of specific food. For the sense of touch, they correspond to, for example, different body parts where a stimulus is applied to, or types or strengths of stimuli.

The measurement unit 13 is connected to the controller 15. The controller 15 provides a measurement signal to the measurement unit 13 based on a program. The measurement unit 13 measures biological information on a user based on the measurement signal input from the controller 15.

The measurement unit 13 is a biological sensor to detect biological information on a user. The biological sensor may be provided at any position as long as the biological sensor is able to detect the biological information on the user. The biological information herein is not something invariable like a fingerprint, and is, for example, information that changes in value according to a state of the user. That is, the biological information is information related to autonomic nerves of the user, that is, information that changes in value regardless of the user's intention.

Specifically, the biological sensor serving as the measurement unit 13 is preferably a pulse wave sensor. The measurement unit 13 detects pulse waves of the user as the biological information. A pulse wave sensor may be, for example, a transmissive photoelectric sensor including a light emitting unit and a light receiving unit. In this case, the pulse wave sensor is, for example, configured such that the light emitting unit and the light receiving unit face each other across a fingertip of a user U, light transmitted through the fingertip is received by the light receiving unit, and a pulse waveform is measured based on the fact that the larger a pressure of a pulse wave, the greater a blood flow. By a second-order differentiation of the pulse waves, a signal corresponding to an R-R interval of an electrocardiogram described after is able to be obtained. However, the pulse wave sensor is not limited to this configuration and may be of any type enabling detection of pulse waves.

The storage 14 is connected to the controller 15. The storage 14 stores various kinds of information. The storage 14 stores activity thresholds in advance, the activity thresholds being activity thresholds to be used when the controller 15 executes a preference determination process. The activity thresholds are thresholds of autonomic nerve activity that have been set in advance correspondingly to multiple types of stimuli. The storage 14 stores multiple activity thresholds correspondingly to predetermined levels of each stimulus. That is, when there are five types of stimuli and five levels for each type of stimulus, the storage 14 stores 25 activity thresholds. In this case, the storage 14 stores them as a two-dimensional activity threshold map composed of the types of stimuli and the levels of the stimuli.

A program for the controller 15 to perform a preference determination is also stored in the storage 14. The storage 14 is an external storage device, such as a hard disk drive (HDD) and a memory, for example.

The controller 15 has a biological information acquisition unit 21, an activity calculation unit 22, and a determination unit 23. The controller 15 is configured composing, for example, an arithmetic circuit, such as a central processing unit (CPU).

The biological information acquisition unit 21 is connected to the measurement unit 13. The biological information acquisition unit 21 controls the measurement unit 13 and causes the measurement unit 13 to detect biological information on the user. The biological information acquisition unit 21 acquires the biological information on the user measured by the measurement unit 13.

The biological information acquisition unit 21 is connected to the activity calculation unit 22. Based on the biological signal acquired by the biological information acquisition unit 21, the activity calculation unit 22 calculates a degree of autonomic nervous activity. Based on the biological signal measured by the measurement unit 13 for each stimulus applied by the stimulation unit 12, the activity calculation unit 22 calculates the degree of autonomic nervous activity. A calculation method for calculating the degree of autonomic nervous activity will be described later.

The activity calculation unit 22 is connected to the determination unit 23. The determination unit 23 determines preference by comparing the degree of autonomic nervous activity calculated by the activity calculation unit 22 with the activity threshold stored in the storage 14. The determination unit 23 determines preference by individually comparing the multiple degrees of autonomic nervous activity calculated according to types of stimuli and levels of the stimuli with the activity thresholds that have set according to the types of stimuli and the levels of the stimuli. The activity thresholds will be described later.

The output unit 16 is capable of outputting a result of control by the controller 15, for example, a result of a determination by the determination unit 23. The output unit 16 is, for example, a display device that displays a video, and a sound output device that outputs sound.

### Degree of Autonomic Nervous Activity

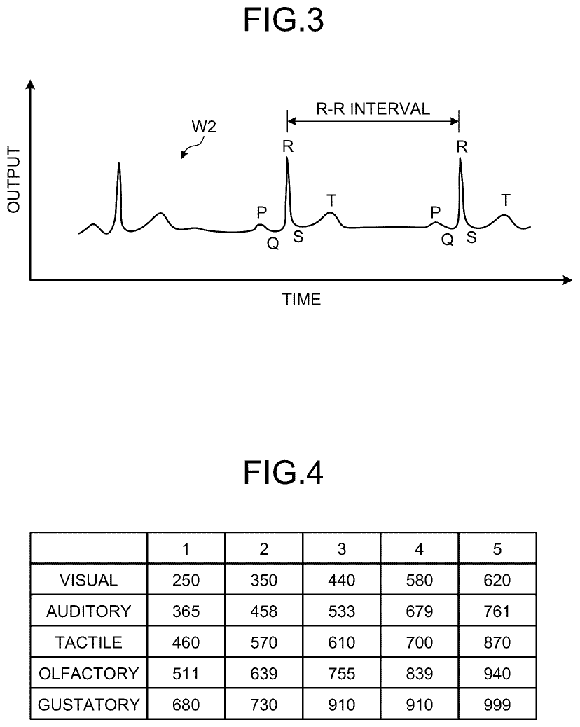

FIG. 2 is a graph illustrating physiological characteristics of a biological signal, and FIG. 3 is a schematic diagram illustrating autonomic nervous activity. The biological signals illustrated in FIG. 2 and FIG. 3 corresponds to an electro-cardiogram related to pulse waves but the biological signals may be biological signals other than brain wave signals.

As illustrated in FIG. 2, a waveform W1 representing an electrocardiogram includes a P wave, a QRS wave, a T wave, and a U wave. For heart rate variation, detection of an R wave that is a peak of the QRS wave is measured as one pulsation.

The electrocardiogram has a waveform with a peak appearing in each predetermined time period, the peak being called an R wave. Pulsations occur by spontaneous ignition of pacemaker cells that are in a sinus node of the heart. Rhythm of the pulses is strongly influenced by both sympathetic nerves and parasympathetic nerves. Sympathetic nerves promote cardiac activity. Parasympathetic nerves reduce cardiac activity. Normally, sympathetic nerves and parasympathetic nerves act antagonistically to each other. At rest or in a state close to being at rest, parasympathetic nerves are dominant. Normally, the pulse rate increases when adrenaline is secreted by excitation of sympathetic nerves and decreases when acetylcholine is secreted by excitation of parasympathetic nerves. Therefore, checking variation in R-R interval on the electrocardiogram is considered to be useful in an autonomic nervous function test.

As illustrated in FIG. 3, in a waveform W2 representing the electrocardiogram, an R-R interval is an interval between chronologically consecutive R waves. For heart rate variation, an R wave that is a peak of a QRS wave of a signal waveform is measured as one pulse. Variation in R wave interval of an electrocardiogram, that is, temporal fluctuation of R-R interval representing a time interval between R waves in FIG. 3 is used as an autonomic nervous index. Many medical institutions have reported validity of using temporal fluctuation of R-R interval as an autonomic nervous index. Fluctuation of R-R interval increases when at rest and decreases when stressed.

Variations in R-R interval include some characteristic fluctuations. One of them is a low frequency component that appears near 0.1 Hz and originates from abnormality in sympathetic nervous system activity associated with feedback regulation of blood pressure in blood vessels. Another one of them is abnormality sympathized with respiration and is a high frequency component reflecting respiratory sinus arrhythmia. This high frequency component reflects direct interference of a respiratory center on vagal preganglionic neurons and pulmonary stretch receptor reflex and baroreceptor reflex upon a pressure change due to respiration, and is considered to be a parasympathetic nervous index that mainly influences the heart. That is, it can be said that among waveform components obtained by the measurement of the fluctuations of R-R interval of the electrocardiogram, a power spectrum of the low frequency component represents activity of the sympathetic nerves and a power spectrum of the high frequency component represents activity of the parasympathetic nerves.

The fluctuations of the electrocardiogram to be input are obtained as differential values of R-R interval values. In this case, if the differential values of the R-R interval are not equally temporally spaced data, the activity calculation unit 22 converts them into equally spaced chronological data using, for example, three-dimensional spline interpolation. The activity calculation unit 22 performs an orthogonal transformation of the differential values of the R-R interval by fast Fourier Transform. The activity calculation unit 22 thereby calculates the power spectrum of the high frequency component and the power spectrum of the low frequency component, the high and low frequency components being of the differential values of the R-R interval values of the electrocardiogram. The activity calculation unit 22 calculates a sum total of the power spectra of the high frequency components as RRHF. The activity calculation unit 22 calculates a sum total of the power spectra of the low frequency components as RRLF. The activity calculation unit 22 calculates a degree of autonomic nervous activity using the following equation.

$$AN = ( C 1 + RRLF ) / ( C 1 + RRHF ) + C 2$$

In the above equation, “AN” is a degree of autonomic nervous activity, “RRHF” is the sum total of power spectra of high frequency components, and “RRLF” is the sum total of power spectra of low frequency components. “C1” and “C2” are fixed values prescribed to reduce divergence of a solution of the degree of autonomic nervous activity AN. The activity calculation unit 22 stores the calculated degree of autonomic nervous activity AN into the storage 14.

### Activity Threshold Map

FIG. 4 is a schematic diagram illustrating an activity threshold map used in preference determination.

The activity threshold map has thresholds for determining a user's preference for a target, for example, whether the user “likes” or “dislikes” the target. The activity threshold map represents activity thresholds for determining preference of a user for a target, in relation to types of stimuli and levels of the stimuli. That is, by comparing calculated degrees of autonomic nervous activity AN with activity thresholds, the determination unit 23 determines preference of a user.

As illustrated in FIG. 4, the activity threshold map has types of stimuli along a vertical axis, levels of the stimuli along a horizontal axis, and activity thresholds respectively set in relation to the types of stimuli and the levels of the stimuli. There are five types of stimuli, that is, the visual stimulus, the auditory stimulus, the tactile stimulus, the olfactory stimulus, and the gustatory stimulus, and five levels for each stimulus. However, the types of stimuli are not limited to this example, and a predetermined number of the five types, that is, the visual stimulus, the auditory stimulus, the tactile stimulus, the olfactory stimulus, and the gustatory stimulus, may be adopted, or the number of levels for each stimulus may be any number other than five.

For example, the stimulation unit 12 applies the visual stimuli to the user by letting the user see videos of “food” of levels 1, 2, 3, 4, and 5 in sequence, and the measurement unit 13 measures a biological signal (brain waves) when each of the videos of “food” of the levels 1, 2, 3, 4, and 5 is seen by the user. The visual levels herein refer to, for example, sizes of the videos, and the level 1 corresponds to a smallest video and the level 5 corresponds to a largest video. The stimulation unit 12 applies the auditory stimuli to the user by letting the user hear sounds of “food” of levels 1, 2, 3, 4, and 5, and the measurement unit 13 measures a biological signal (brain waves) when each of the sounds of “food” of the levels 1, 2, 3, 4, and 5 is heard by the user. The auditory levels herein refer to, for example, loudness levels of the sound. The sounds of “food” are sounds of names of the food and/or sounds of cooking the food.

The stimulation unit 12 applies the tactile stimuli to the user by, for example, generating temperatures of “food” of levels 1, 2, 3, 4, and 5 for the user by means of a heating device or a cooling device, and the measurement unit 13 measures a biological signal (brain waves) when each of the temperatures of “food” of the levels 1, 2, 3, 4, and 5 is generated. As to the tactile levels herein, for example, a temperature that is the same as the average temperature of specific “food” is the level 5, and as the temperature differs from this value, the level decreases to the levels 4, 3, 2, and 1 in order. Or the tactile levels refer to, for example, contact areas or contact time periods. The stimulation unit 12 applies the olfactory stimuli by letting the user smell smells (fragrances) of “food” of levels 1, 2, 3, 4, and 5 by means of a device including a dedicated cartridge that discharges smells and fragrances, and the measurement unit 13 measures a biological signal (brain waves) when each of the smells of “food” of the levels 1, 2, 3, 4, and 5 is smelt by the user. The olfactory levels herein refer to, for example, strength levels of the smells (fragrances). The stimulation unit 12 applies the gustatory stimuli by letting the user eat “food” of levels 1, 2, 3, 4, and 5 in sequence by means of a device including a dedicated cartridge that discharges tastes, and the measurement unit 13 measures a biological signal (brain waves) when each of “food” of the levels 1, 2, 3, 4, and 5 is eaten by the user. The gustatory levels herein refer to, for example, strength levels or amounts of a taste.

Some examples of the visual, auditory, tactile, olfactory, and gustatory stimuli to be applied by the stimulation unit 12 have been described above, but methods of applying these stimuli are not to be limited to these examples. The stimulation unit 12 may apply stimuli to a user as visual, auditory, tactile, olfactory, and gustatory stimuli by letting the user see, hear, touch, smell, and eat the real target.

Specifically, the stimulation unit 12 applies the tactile stimuli to the user by letting the user touch real “food” of levels 1, 2, 3, 4, and 5 in sequence, and the measurement unit 13 measures a biological signal (brain waves) when “food” of each of the levels 1, 2, 3, 4, and 5 is touched by the user. The tactile levels herein refer to, for example, contact areas or contact time periods. The stimulation unit 12 applies the olfactory stimuli to the user by letting the user smell smells (fragrances) of real “food” of levels 1, 2, 3, 4, and 5, and the measurement unit 13 measures a biological signal (brain waves) when each of the smells of “food” of the levels 1, 2, 3, 4, and 5 is smelt by the user. The olfactory levels herein refer to, for example, strength levels of the smells (fragrances). The stimulation unit 12 applies the gustatory stimuli to the user by letting the user eat real “food” of levels 1, 2, 3, 4, and 5 in sequence, and the measurement unit 13 measures a biological signal (brain waves) when “food” of each of the levels 1, 2, 3, 4, and 5 is eaten by the user. The gustatory levels herein refer to, for example, strength levels or amounts of a taste.

The biological information acquisition unit 21 acquires biological signals measured by the measurement unit 13, and the activity calculation unit 22 calculates, based on the biological signals acquired by the biological information acquisition unit 21, degrees of autonomic nervous activity. The determination unit 23 compares the degrees of autonomic nervous activity calculated by the activity calculation unit 22 to the activity thresholds recorded in the activity threshold map. In this case, for the 25 items corresponding to the relation between the types of stimuli and the levels of the stimuli, the degrees of autonomic nervous activity of the user and the activity thresholds are compared to each other. If the degree of autonomic nervous activity of the user is larger than the activity threshold for each of the items, it is determined that the user “likes” the “food”. If the degrees of autonomic nervous activity of the user are larger than the activity thresholds for all the items, it is determined that the user “likes” the “food”. On the contrary, if the degrees of autonomic nervous activity of the user are equal to or less than the activity thresholds for all the items, it is determined that the user “dislikes” the “food”.

### Modified Examples of Activity Threshold Map

The activity threshold map may have, instead of the types of stimuli, a predetermined number of different senses of one of the sense organs corresponding to the five senses, along the vertical axis.

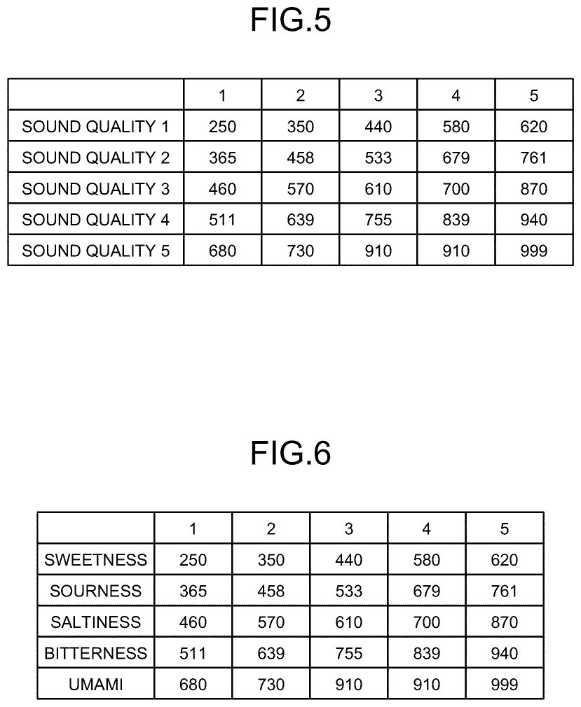

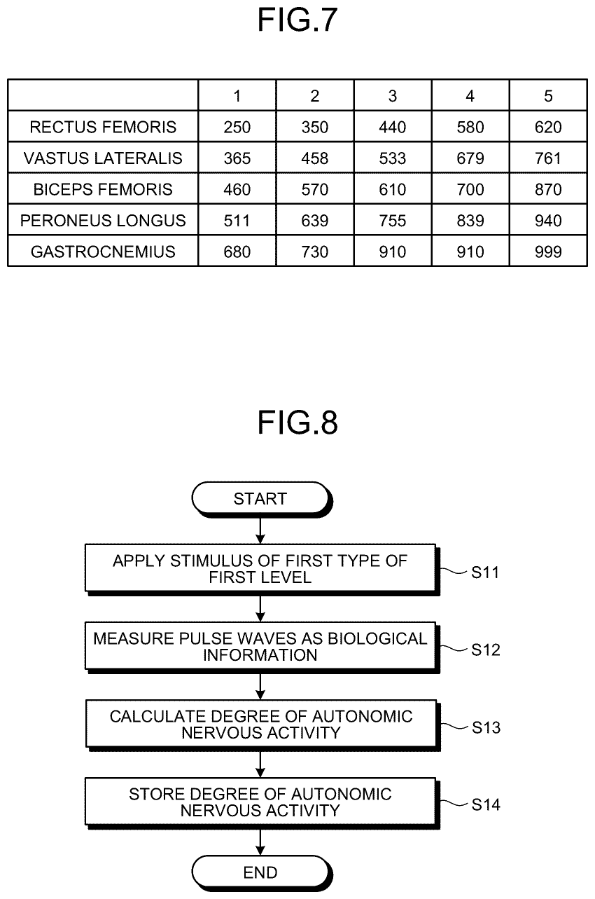

FIG. 5 is a schematic diagram illustrating a first modified example of the activity threshold map, FIG. 6 is a schematic diagram illustrating a second modified example of the activity threshold map, and FIG. 7 is a schematic diagram illustrating a third modified example of the activity threshold map.

For example, as illustrated in FIG. 5, an activity threshold map may have multiple senses for hearing that are different sound qualities along the vertical axis, and levels of these sound qualities along the horizontal axis. Sound qualities 1, 2, 3, 4, and 5 are different sound qualities for a specific “music”. Levels 1, 2, 3, 4, and 5 are sound volume levels.

Furthermore, as illustrated in FIG. 6, an activity threshold map may have sweetness, sourness, saltiness, bitterness, and umami, which are multiple senses for taste along the vertical axis, and taste levels along the horizontal axis. Levels 1, 2, 3, 4, and 5 are strength levels of the tastes.

Furthermore, as illustrated in FIG. 7, an activity threshold map may have different body parts or exercise types that are multiple senses for touch along the vertical axis and tactile levels along the horizontal axis. For example, types of stimuli may include “rectus femoris”, “vastus lateralis”, “biceps femoris”, “peroneus longus muscles”, and “gastrocnemius muscles”, or “one-leg standing”, “jogging”, “thigh raising”, “leg lunging”, and “squatting”. Levels 1, 2, 3, 4, and 5 are strength levels of the stimuli. The strength levels of the stimuli herein refer to time periods of “one-leg standing”, time periods or distances of “jogging”, and time periods or the numbers of times of “thigh raising”, “leg lunging”, and “squatting”. As to how the stimuli are applied to a user, for example, the user is notified of the time periods and the number of times by sound through a speaker and caused to perform the exercises for the time periods and the number of times, and the degree of activity is then recorded.

The thresholds in the activity threshold map are preferably obtained in advance by experiments. Degrees of autonomic nervous activity are calculated by applying stimuli of multiple items in an activity threshold map to multiple users, and their preference for a target such as “food” is confirmed. The thresholds in the activity threshold map are set by association between the degrees of autonomic nervous activity and the preference for the target.

### Preference Determination Method

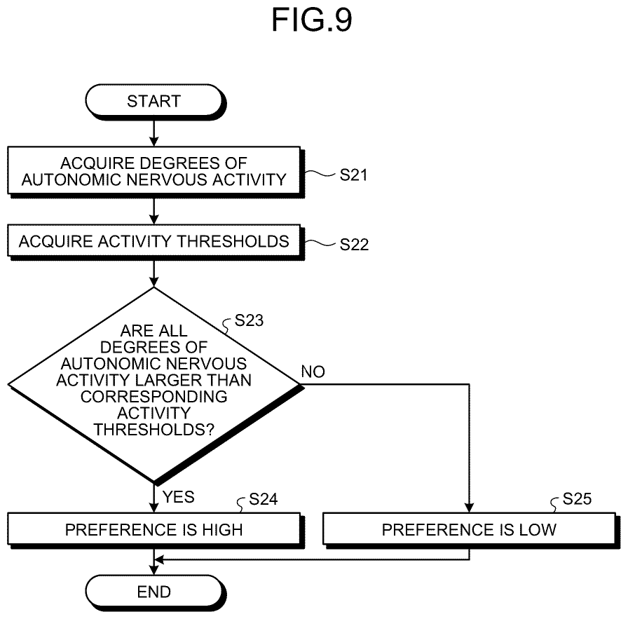

FIG. 8 is a flowchart illustrating a method of calculating a degree of activity, and FIG. 9 is a flowchart illustrating a preference determination method.

As illustrated in FIG. 1 and FIG. 8, at Step S11, the stimulation unit 12 applies a stimulus of a first type of a first level to a user. The stimulus of the first type of the first level herein refers to the visual stimulus of the level 1 in the activity threshold map of FIG. 4. At Step S12, the measurement unit 13 measures pulse waves as biological information on the user, and the biological information acquisition unit 21 acquires the pulse waves that are the biological information on the user measured by the measurement unit 13. At Step S13, the activity calculation unit 22 calculates a degree of autonomic nervous activity based on the pulse waves that are the biological information on the user acquired by the biological information acquisition unit 21. At Step S14, the activity calculation unit 22 stores the calculated degree of autonomic nervous activity into the storage 14.

Through Step S11 to Step S14, the degree of autonomic nervous activity of the user upon applying the stimulus of the first type of the first level to the user is calculated. Similarly, by a method similar to Step S11 to Step S14, the degrees of autonomic nervous activity of the user upon sequentially applying the remaining stimuli of the five types of the five levels to the user are respectively calculated.

As illustrated in FIG. 1 and FIG. 9, at Step S21, the determination unit 23 acquires the degrees of autonomic nervous activity of the user calculated for all the items (the 25 items in the activity threshold map of FIG. 4) stored in the storage 14. At Step S22, the determination unit 23 acquires the corresponding activity threshold map (activity thresholds) that has been stored in the storage 14.

At Step S23, the determination unit 23 determines whether or not the degrees of autonomic nervous activity of the user for all the items are larger than the corresponding activity thresholds in the activity threshold map. In a case where the determination unit 23 determines that the degrees of autonomic nervous activity of the user for all the items are larger than the corresponding activity thresholds in the activity threshold map (Yes), the determination unit 23 determines at Step S24 that the user's preference for the target is high, that is, the user likes the target. On the contrary, in a case where the determination unit 23 determines that some of the degrees of autonomic nervous activity of the user for the items are not larger than the corresponding activity thresholds in the activity threshold map (No), the determination unit 23 determines at Step S25 that the user's preference for the target is low, that is, the user does not like the target.

### Second Embodiment

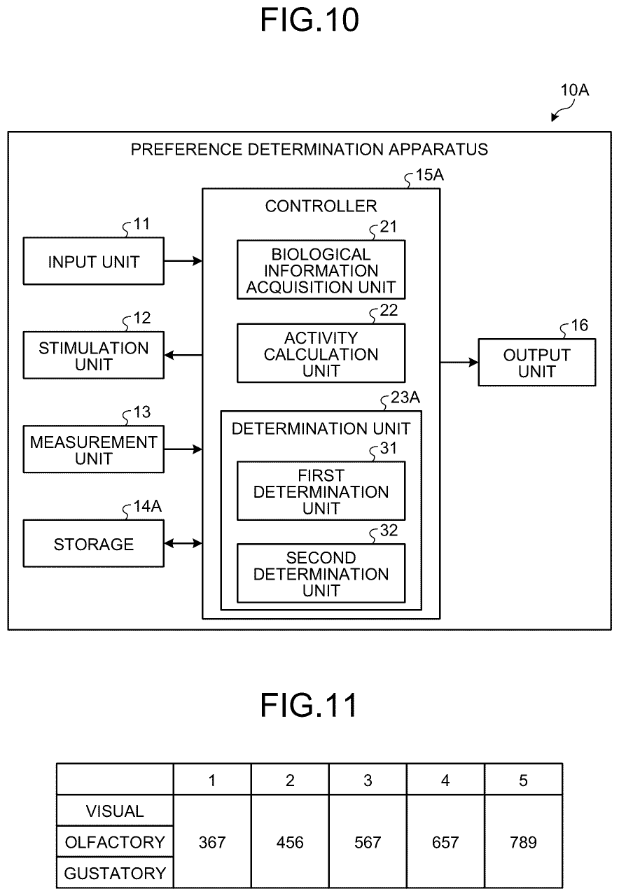

FIG. 10 is a block diagram illustrating a preference determination apparatus according to a second embodiment. The same reference signs will be assigned to members having functions similar to those of the first embodiment described above, and detailed description thereof will be omitted.

### Preference Determination Apparatus

As illustrated in FIG. 10, a preference determination apparatus 10A includes an input unit 11, a stimulation unit 12, a measurement unit 13, a storage 14A, a controller 15A, and an output unit 16. The input unit 11, the stimulation unit 12, the measurement unit 13, and the output unit 16 are similar to those of the first embodiment.

The storage 14A stores therein activity thresholds in advance, the activity thresholds being activity thresholds to be used when the controller 15A executes a preference determination process. The activity thresholds are thresholds of autonomic nervous activity that have set in advance correspondingly to multiple types of stimuli. The storage unit 14A stores multiple activity thresholds for predetermined levels of each stimulus. In this case, the storage 14A stores a first activity threshold map (similar to that according to the first embodiment) to be used in determination upon sequentially applying one type of stimulus to a user, and a second activity threshold map to be used in determination upon simultaneously applying multiple types of stimuli to the user.

The controller 15A has a biological information acquisition unit 21, an activity calculation unit 22, and a determination unit 23A. The determination unit 23A has a first determination unit 31 and a second determination unit 32. The biological information acquisition unit 21 and the activity calculation unit 22 are similar to those according to the first embodiment.

The first determination unit 31 and the second determination unit 32 of the determination unit 23A determines preference by comparing the degrees of autonomic nervous activity calculated by the activity calculation unit 22 to the activity thresholds stored in the storage 14A. Similarly to the determination unit 23 according to the first embodiment, the first determination unit 31 determines preference by comparing the degrees of autonomic nervous activity sequentially calculated upon sequentially applying one type of stimulus to a user with the corresponding activity thresholds in all of the types of stimuli and the levels of the stimuli. The second determination unit 32 determines preference by comparing the degrees of autonomic nervous activity calculated upon simultaneously applying the multiple types of stimuli to the user with the corresponding activity thresholds set for applying the multiple types of stimuli.

That is, the stimulation unit 12 applies multiple stimuli to a user at predetermined intervals, the measurement unit 13 sequentially measures biological signals (brain waves) of the user, and the biological information acquisition unit 21 acquires these biological signals. The activity calculation unit 22 sequentially calculates the degree of autonomic nervous activity based on the biological signal for each stimulus applied by the stimulation unit 12, and the first determination unit 31 makes a determination of preference of the user by comparing the multiple degrees of autonomic nervous activity calculated by the activity calculation unit 22 with the first activity threshold map stored in the storage 14A.

Furthermore, the stimulation unit 12 simultaneously applies the multiple stimuli to the user, the measurement unit 13 measures biological signals (brain waves) of the user, and the biological information acquisition unit 21 acquires these biological signals. The activity calculation unit 22 sequentially calculates the degrees of autonomic nervous activity based on the biological signals for the respective multiple stimuli applied by the stimulation unit 12, and the second determination unit 32 makes a determination of preference of the user by comparing the degrees of autonomic nervous activity calculated by the activity calculation unit 22 with the second activity threshold map stored in the storage 14A.

Based on a result of the determination by the first determination unit 31 and a result of the determination by the second determination unit 32, the determination unit 23A finally determines preference of the user.

### Activity Threshold Map

FIG. 11 is a schematic diagram illustrating a second activity threshold map used in preference determination. The first activity threshold map is similar to that according to the first embodiment.

As illustrated in FIG. 11, the second activity threshold map has types of stimuli along the vertical axis, levels of the stimuli along the horizontal axis, and activity thresholds respectively set for the levels of the stimuli. There are three types of stimuli, that is, the visual, olfactory, and gustatory stimuli, and five levels for the stimuli. However, the types of stimuli are not limited to these three, and a predetermined number of five types, that is, the visual, auditory, tactile, olfactory, and gustatory stimuli, may be adopted, or the number of the levels of the stimuli may be other than five.

For example, the stimulation unit 12 simultaneously applies the visual stimulus, the olfactory stimulus, and the gustatory stimulus to the user. That is, the stimulation unit 12 lets the user see a video of “food” of a level 1, lets the user smell a smell (fragrance) of “food” of a level 1, and lets the user eat “food” of a level 1. The measurement unit 13 measures a biological signal (brain waves) of the user upon simultaneously applying of the visual stimulus, the olfactory stimulus, and the gustatory stimulus, of the level 1. A similar process is performed for each of levels 2, 3, 4, and 5 for the visual stimulus, the olfactory stimulus, and the gustatory stimulus.

The measurement unit 13 measures the biological signals (brain waves) of the user for each of the levels 1, 2, 3, 4, and 5 upon simultaneously applying the three types of stimuli. The biological information acquisition unit 21 acquires the biological signals measured by the measurement unit 13, and the activity calculation unit 22 calculates degrees of autonomic nervous activity based on the biological signals acquired by the biological information acquisition unit 21. The second determination unit 32 compares the degrees of autonomic nervous activity calculated by the activity calculation unit 22 with the activity thresholds recorded in the second activity threshold map. In this case, for the five items of the levels of stimuli, the degrees of autonomic nervous activity of the user and the second activity thresholds are compared to each other.

### Preference Determination Method

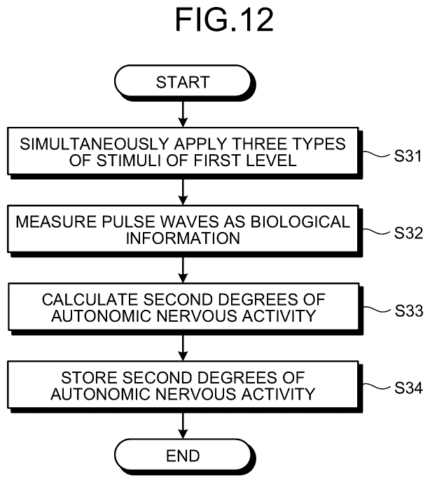

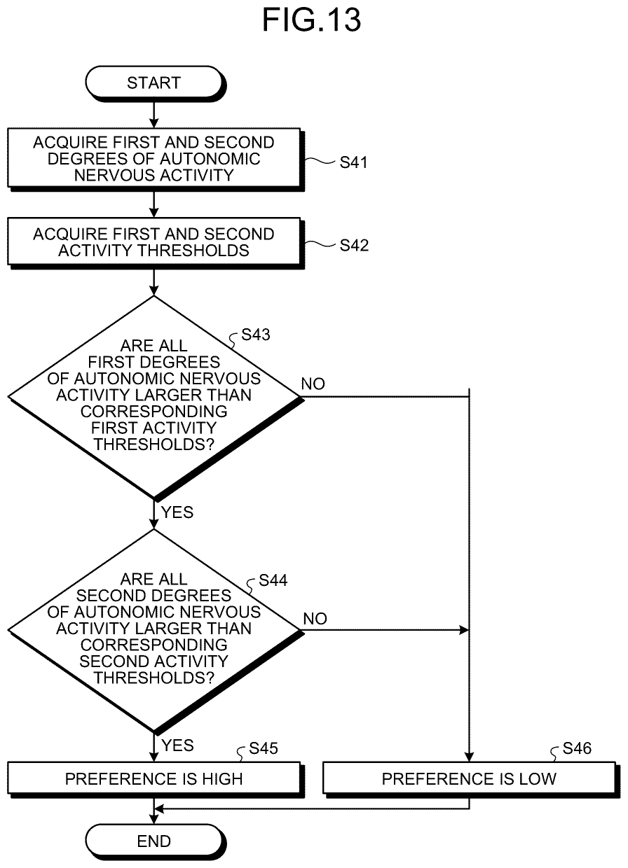

FIG. 12 is a flowchart illustrating a method of calculating a degree of activity, and FIG. 13 is a flowchart illustrating a preference determination method.

As illustrated in FIG. 10 and FIG. 12, at Step S31, the stimulation unit 12 simultaneously applies three types of stimuli of a first level to a user. The three types of stimuli of the first level herein refer to the visual, olfactory, and gustatory stimuli of the level 1 in the second activity threshold map of FIG. 11. At Step S32, the measurement unit 13 measures pulse waves as biological information on the user, to whom the three types of stimuli have been simultaneously applied, and the biological information acquisition unit 21 acquires the pulse waves as the biological information on the user measured by the measurement unit 13. At Step S33, the activity calculation unit 22 calculates a second degree of autonomic nervous activity based on the pulse waves that are the biological information on the user acquired by the biological information acquisition unit 21. At Step S34, the activity calculation unit 22 stores the calculated second degree of autonomic nervous activity into the storage unit 14A.

Through Step S31 to Step S34, the degree of autonomic nervous activity of the user upon applying the stimuli of the first level to the user is calculated. Similarly, by a method similar to Step S31 to Step S34, the degrees of autonomic nervous activity of the user upon simultaneously applying the stimuli of each of the remaining four levels to the user are calculated.

As illustrated in FIG. 10 and FIG. 13, at Step S41, the first determination unit 31 acquires first degrees of autonomic nervous activity of the user calculated for all the items (the 25 items in the activity map of FIG. 4) stored in the storage 14A. Furthermore, the second determination unit 32 acquires second degrees of autonomic nervous activity calculated for all the items (the five items in the activity map of FIG. 11) stored in the storage 14A. At Step S42, the first determination unit 31 acquires the corresponding first activity threshold map (first activity thresholds) that has been stored in the storage 14A. Furthermore, the second determination unit 32 acquires the corresponding second activity threshold map (second activity thresholds) that has been stored in the storage 14A.

At Step S43, the first determination unit 31 determines whether or not the first degrees of autonomic nervous activity of the user for all the items are larger than the corresponding first activity thresholds in the first activity threshold map. In a case where the first determination unit 31 determines that the first degrees of autonomic nervous activity of the user for all the items are larger than the corresponding first activity thresholds in the first activity threshold map (Yes), the first determination unit 31 proceeds to Step S44.

At Step S44, the second determination unit 32 determines whether or not the second degrees of autonomic nervous activity of the user for all the items are larger than the corresponding second activity thresholds in the second activity threshold map. In a case where the second determination unit 32 determines that the second degrees of autonomic nervous activity of the user for all the items are larger than the corresponding second activity thresholds in the second activity threshold map (Yes), the second determination unit 32 determines at Step S45 that the user's preference for the target is high, that is, the user likes the target.

On the contrary, in a case where the first determination unit 31 determines at Step S43 that some of the first degrees of autonomic nervous activity of the user for the items are not larger than the corresponding first activity thresholds in the first activity threshold map (No), the first determination unit 31 determines at Step S46 that the user's preference for the target is low, that is, the user does not like the target. Furthermore, in a case where the second determination unit 32 determines at Step S44 that some of the second degrees of autonomic nervous activity of the user for the items are not larger than the corresponding second activity thresholds in the second activity threshold map (No), the second determination unit 32 determines at Step S46 that the user's preference for the target is low, that is, the user does not like the target.

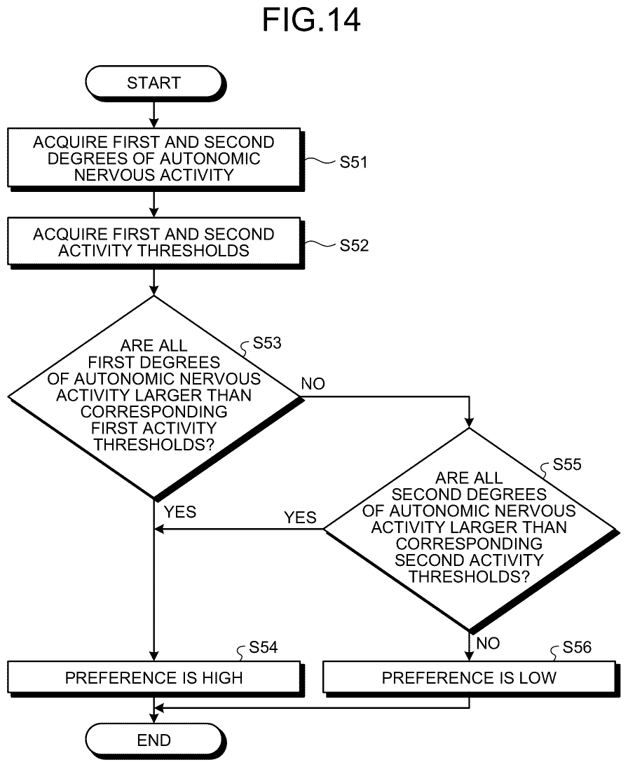

The determination method by the determination unit 23A is not limited to the above described determination method. FIG. 14 is a flowchart illustrating a modified example of the preference determination method.

As illustrated in FIG. 10 and FIG. 14, at Step S51, the first determination unit 31 acquires the first degrees of autonomic nervous activity of the user calculated for all of items (the 25 items in the activity map of FIG. 4) stored in the storage 14A. Furthermore, the second determination unit 32 acquires the second degrees of autonomic nervous activity of the user calculated for all the items (the five items in the activity map of FIG. 11) stored in the storage 14A. At Step S52, the first determination unit 31 acquires the corresponding first activity threshold map (first activity thresholds) that has been stored in the storage 14A. Furthermore, the second determination unit 32 acquires the corresponding second activity threshold map (second activity thresholds) that has been stored in the storage 14A.

At Step S53, the first determination unit 31 determines whether or not the first degrees of autonomic nervous activity of the user for all the items are larger than the corresponding first activity thresholds in the first activity threshold map. In a case where the first determination unit 31 determines that the first degrees of autonomic nervous activity of the user for all the items are larger than the corresponding first activity thresholds in the first activity threshold map (Yes), the first determination unit 31 determines at Step S54 that the user's preference for the target is high, that is, the user like the target.

On the contrary, in a case where the first determination unit 31 determines at Step S53 that some of the first degrees of autonomic nervous activity of the user for the items are not larger than the corresponding first activity thresholds in the first activity threshold map (No), the first determination unit 31 proceeds to Step S55. At Step S55, the second determination unit 32 determines whether or not the second degrees of autonomic nervous activity of the user for all the items are larger than the corresponding second activity thresholds in the second activity threshold map. In a case where the second determination unit 32 determines that the second degrees of autonomic nervous activity of the user for all the items are larger than the corresponding second activity thresholds in the second activity threshold map (Yes), the second determination unit 32 determines at Step S54 that the user's preference for the target is high, that is, the user likes the target.

On the contrary, in a case where the second determination unit 32 determines at Step S55 that some of the second degrees of autonomic nervous activity of the user for the items are not larger than the corresponding second activity thresholds in the second activity threshold map (No), the second determination unit 32 determines at Step S56 that the user's preference for the target is low, that is, the user does not like the target.

### Functions and Effects of Embodiments

A preference determination apparatus according to the present application includes: a stimulation unit (12) configured to apply a visual stimulus, a tactile stimulus, and an auditory stimulus; an activity calculation unit (22) configured to calculate degrees of autonomic nervous activity based on biological signals for the multiple stimuli respectively; a storage (14A) configured to store thresholds of autonomic nervous activity set in advance for the visual stimulus, the tactile stimulus, and the auditory stimulus respectively; and a determination unit (23A) configured to determine preference by comparing the degrees of autonomic nervous activity calculated by the activity calculation unit with the corresponding thresholds stored in the storage, wherein the stimulation unit (12) is further configured to apply each of the multiple stimuli at predetermined intervals, the activity calculation unit (22) is further configured to calculate the degree of autonomic nervous activity based on the biological signal for each of the multiple stimuli applied by the stimulation unit (12), the storage (14A) is further configured to store the threshold of autonomic nervous activity for each of the multiple stimuli, and the determination unit (23A) has a first determination unit (31) configured to determine the preference by comparing the multiple degrees of autonomic nervous activity calculated by the activity calculation unit (22) with the corresponding thresholds stored in the storage (14A), the stimulation unit (12) is further configured to apply the multiple stimuli simultaneously, the activity calculation unit (22) is further configured to calculate the degree of autonomic nervous activity based on the biological signal for the multiple stimuli simultaneously applied by the stimulation unit (12), the storage (14A) is further configured to store the threshold of autonomic nervous activity for the multiple stimuli simultaneously applied, and the determination unit (23A) has a second determination unit (32) configured to determine the preference by comparing the degree of autonomic nervous activity calculated by the activity calculation unit (22) with the corresponding threshold stored in the storage (14A), and the determination unit (23A) is further configured to determine that the preference is high when each of the degrees of autonomic nervous activity calculated based on the biological signal for each of the stimuli applied by the stimulation unit (12) is larger than each of the corresponding thresholds of autonomic nervous activity stored in the storage (14A), and when the degree of autonomic nervous activity calculated based on the biological signal for the stimuli simultaneously applied by the stimulation unit (12) is larger than the corresponding threshold of autonomic nervous activity stored for the multiple stimuli simultaneously applied.

Therefore, the preference of the user is able to be determined by comparing the degrees of autonomic nervous activity and the activity thresholds to each other, and the preference of the user is able to be determined easily using sense organs of humans.

A preference determination apparatus according to the present application includes: a stimulation unit (12) configured to apply a visual stimulus, a tactile stimulus, and an auditory stimulus; an activity calculation unit (22) configured to calculate degrees of autonomic nervous activity based on biological signals for the multiple stimuli respectively; a storage (14A) configured to store thresholds of autonomic nervous activity set in advance for the visual stimulus, the tactile stimulus, and the auditory stimulus respectively; and a determination unit (23A) configured to determine preference by comparing the degrees of autonomic nervous activity calculated by the activity calculation unit (22) with the corresponding thresholds stored in the storage (14A), wherein the stimulation unit (12) is further configured to apply each of the multiple stimuli at predetermined intervals, the activity calculation unit (22) is further configured to calculate the degree of autonomic nervous activity based on the biological signal for each of the multiple stimuli applied by the stimulation unit (12), the storage (14A) is further configured to store the threshold of autonomic nervous activity for each of the multiple stimuli, and the determination unit (23A) has a first determination unit (31) configured to determine the preference by comparing the multiple degrees of autonomic nervous activity calculated by the activity calculation unit (22) with the corresponding thresholds stored in the storage (14A), the stimulation unit (12) is further configured to apply the multiple stimuli simultaneously, the activity calculation unit (22) is further configured to calculate the degree of autonomic nervous activity based on the biological signal for the multiple stimuli simultaneously applied by the stimulation unit (12), the storage (14A) is further configured to store the threshold of autonomic nervous activity for the multiple stimuli simultaneously applied, and the determination unit (23A) has a second determination unit (32) configured to determine the preference by comparing the degree of autonomic nervous activity calculated by the activity calculation unit (22) with the corresponding threshold stored in the storage (14A), and the determination unit (23A) is further configured to determine that the preference is high when any of the degrees of autonomic nervous activity calculated based on the biological signal for each of the stimuli applied by the stimulation unit (12) is not larger than the corresponding threshold of autonomic nervous activity stored in the storage (14A), and when the degree of autonomic nervous activity calculated based on the biological signal for the stimuli simultaneously applied by the stimulation unit (12) is larger than the corresponding threshold of autonomic nervous activity stored for the multiple stimuli simultaneously applied.

Therefore, the preference of the user is able to be determined by comparing the degrees of autonomic nervous activity and the activity thresholds to each other, and the preference of the user is able to be determined easily using sense organs of humans.

The preference determination apparatuses according to the present application have been described thus far, but implementation in various different modes other than the above described embodiments is possible.

Each component of the preference determination apparatuses has been functionally and/or conceptually illustrated in the drawings, and is not necessarily configured physically as illustrated in the drawings. That is, the specific form of each apparatus is not limited to the one illustrated in the drawings, and all or part of each apparatus may be functionally or physically separated or integrated in any units according to, for example, the processing load on the apparatus and the use situation of the apparatus.

The configurations of the preference determination apparatuses are each implemented as software by a program loaded into a memory, for example. With respect to the embodiments, functional blocks implemented by cooperation among these pieces of hardware or pieces of software have been described above. That is, these functional blocks may be implemented in any of various forms, by hardware only, software only, or a combination of hardware and software.

According to the present application, an effect of enabling determination of preference using sense organs of a person is able to be achieved.

The preference determination apparatus, the preference determination method, and the non-transitory storage medium according to the present application is able to be available for a processing apparatus like a computer.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A preference determination apparatus, comprising:
a stimulation unit configured to apply a visual stimulus, a tactile stimulus, and an auditory stimulus;
an activity calculation unit configured to calculate degrees of autonomic nervous activity based on biological signals for the multiple stimuli respectively;
a storage configured to store thresholds of autonomic nervous activity set in advance for the visual stimulus, the tactile stimulus, and the auditory stimulus respectively; and
a determination unit configured to determine preference by comparing the degrees of autonomic nervous activity calculated by the activity calculation unit with the corresponding thresholds stored in the storage, wherein
the stimulation unit is further configured to apply each of the multiple stimuli at predetermined intervals, the activity calculation unit is further configured to calculate the degree of autonomic nervous activity based on the biological signal for each of the multiple stimuli applied by the stimulation unit, the storage is further configured to store the threshold of autonomic nervous activity for each of the multiple stimuli, and the determination unit has a first determination unit configured to determine the preference by comparing the multiple degrees of autonomic nervous activity calculated by the activity calculation unit with the corresponding thresholds stored in the storage,
the stimulation unit is further configured to apply the multiple stimuli simultaneously, the activity calculation unit is further configured to calculate the degree of autonomic nervous activity based on the biological signal for the multiple stimuli simultaneously applied by the stimulation unit, the storage is further configured to store the threshold of autonomic nervous activity for the multiple stimuli simultaneously applied, and the determination unit has a second determination unit configured to determine the preference by comparing the degree of autonomic nervous activity calculated by the activity calculation unit with the corresponding threshold stored in the storage, and
the determination unit is further configured to determine that the preference is high when each of the degrees of autonomic nervous activity calculated based on the biological signal for each of the stimuli applied by the stimulation unit is larger than each of the corresponding thresholds of autonomic nervous activity stored in the storage, and when the degree of autonomic nervous activity calculated based on the biological signal for the stimuli simultaneously applied by the stimulation unit is larger than the corresponding threshold of autonomic nervous activity stored for the multiple stimuli simultaneously applied.

2. A preference determination method, comprising:
applying a visual stimulus, a tactile stimulus, and an auditory stimulus;
calculating degrees of autonomic nervous activity based on biological signals for the applied stimulus;
storing thresholds of autonomic nervous activity that have been set in advance for the visual stimulus, the tactile stimulus, and the auditory stimulus respectively; and
determining preference by comparing the calculated degrees of autonomic nervous activity with the corresponding stored thresholds, further comprises:
applying each of the multiple stimuli at predetermined intervals;
calculating the degree of autonomic nervous activity based on the biological signal for each of the multiple stimuli applied by the stimulation unit;
storing the threshold of autonomic nervous activity for each of the multiple stimuli; and
determining the preference by comparing the calculated multiple degrees of autonomic nervous activity with the corresponding stored thresholds, and
applying the multiple stimuli simultaneously;
calculating the degree of autonomic nervous activity based on the biological signals for the multiple stimuli simultaneously applied;
storing the threshold of autonomic nervous activity for the multiple stimuli simultaneously applied; and
determining the preference by comparing the calculated degree of autonomic nervous activity with the corresponding stored threshold, and
determining that the preference is high when each of the degrees of autonomic nervous activity calculated based on the biological signal for each of the applied stimuli is larger than each of the corresponding thresholds of autonomic nervous activity stored in the storage, and also when the degree of autonomic nervous activity calculated based on the biological signal for the stimuli simultaneously applied is larger than the corresponding threshold of autonomic nervous activity stored for the multiple stimuli simultaneously applied.

3. A non-transitory storage medium that stores a program that causes a computer, which operates as a preference determination apparatus, to execute:
a step of applying a visual stimulus, a tactile stimulus, and an auditory stimulus;
a step of calculating degrees of autonomic nervous activity based on biological signals for the applied stimulus;
a step of storing thresholds of autonomic nervous activity that have been set in advance for the visual stimulus, the tactile stimulus, and the auditory stimulus respectively; and
a step of determining preference by comparing the calculated degrees of autonomic nervous activity with the corresponding stored thresholds, further comprises:
a step of applying each of the multiple stimuli at predetermined intervals;
a step of calculating the degree of autonomic nervous activity based on the biological signal for each of the multiple stimuli applied by the stimulation unit;
a step of storing the threshold of autonomic nervous activity for each of the multiple stimuli; and
a step of determining the preference by comparing the calculated multiple degrees of autonomic nervous activity with the corresponding stored thresholds, and
a step of applying the multiple stimuli simultaneously;
a step of calculating the degree of autonomic nervous activity based on the biological signals for the multiple stimuli simultaneously applied;
a step of storing the threshold of autonomic nervous activity for the multiple stimuli simultaneously applied; and
a step of determining the preference by comparing the calculated degree of autonomic nervous activity with the corresponding stored threshold, and
a step of determining that the preference is high when each of the degrees of autonomic nervous activity calculated based on the biological signal for each of the applied stimuli is larger than each of the corresponding thresholds of autonomic nervous activity stored in the storage, and also when the degree of autonomic nervous activity calculated based on the biological signal for the stimuli simultaneously applied is larger than the corresponding threshold of autonomic nervous activity stored for the multiple stimuli simultaneously applied.

4. The preference determination apparatus according to claim 1, wherein
the visual stimulus includes visual stimuli of multiple levels of different sizes;
the tactile stimulus includes tactile stimuli of multiple levels of different sizes of a contact area or different periods of a contact time with a contacted object; and
the auditory stimulus includes auditory stimuli of multiple levels of different loudness levels.

5. The preference determination apparatus according to claim 1, wherein the stored threshold of autonomic nervous activity for each of the multiple stimuli is calculated by applying each of the multiple stimuli at predetermined intervals to multiple users in advance.
